(19) United States
(12) Patent Application Publication
(10) Pub. No.: US 2022/0317112 A1
(43) Pub. Date: Oct. 6, 2022

(54) CULTURE DEVICE

(71) Applicant: CARCINOTECH LTD, Midlothian (GB)

(72) Inventor: Ishani MALHOTRA, Midlothian (GB)

(21) Appl. No.: 17/596,879

(22) PCT Filed: Jun. 19, 2020
(86) PCT No.: PCT/EP2020/067247

§ 371 Date: Dec. 20, 2021

(30) Foreign Application Priority Data
Jun. 20, 2019 (GB) 1908841.8

(51) Int. Cl. G01N 33/50; C12N 5/09; C12M 1/36; C12M 1/12; B33Y 80/00

(52) U.S. Cl. CPC G01N 33/5011; C12N 5/0693; C12M 41/48; C12M 25/14; B33Y 80/00; C12N 2500/02; C12N 2513/00; C12N 2533/54; C12N 2533/52; C12N 2533/72; C12N 2533/74

(57) ABSTRACT

Disclosed is a device for the culture of cells, which device is able to support and/or maintain the cells within an environment which mimics one or more in vivo environmental condition(s). Using these devices, cells can be cultured or maintained under conditions which ensure that the cells behave and respond substantially as they would in vivo. Further, the cells can be stimulated or exposed to exogenous agents (drugs and the like) and any response determined to be one which is indicative of an in vivo response.

### BACKGROUND

Cancer stem cells (CSCs) are tumour cells which have the property of stem cells; in particular CSCs have the capacity to self-renew, they give rise to different cell types in a tumour population, they grow uncontrollably, they display plasticity, they can cause initiation, metastasis, and relapse of cancer, they can be dormant for a long time before acting up, they do not undergo apoptosis and are not affected by any form of cancer therapeutics. As such, the tumourigenic profile and stem cell-like attributes make a fatal combination.

Cancer stem cells usually divide asymmetrically, this leads to the daughter cells also being CSCs and causes a growth in the tumour. Stem cells reside in specialized microenvironments called niches which regulate and modulate the properties of these cells. Cancer stem cell niches help maintain the CSC phenotype and shield combined, this plays and important part in tutor initiation (Plaks, et al., 2015).

Cancer stem cell niches are part of the tumour microenvironment and may comprise various types of cells that all play a part in maintaining tumour progression and growth. The CSC niche cell types may include:
1. Cancer-associated fibroblasts(CAFs);
2. Mesenchymal stem cells(MSCs);
3. Tumour-associated inflammatory cells;
4. Non-CSC tumour cells; and
5. Endothelial cells.

A CSC niche may also comprise components of the extracellular matrix (ECM), hypoxic conditions and other factors and signals that result from cell-cell contact.

dishes or even on a gel matrix. More recent studies may also involve the use of scaffolds to study 3D structures of cancers and their interactions with other cells. These models are good for cancer research but provide little insight into the role of processes like EMT (Epithelial-mesenchymal transition) and the mechanisms which underpin cancer stem cell growth and tumour initiation. For example, some CSCs are non-adherent, this makes it difficult to grow them on simple culture plates.

Accordingly, it is essential that CSC research is provided with tools, methods and devices which mimic the in vivo conditions of a CSC niche.

### SUMMARY

The present disclosure provides a device for the culture of cells, which device is able to support and/or maintain the cells within an environment which mimics one or more in vivo environmental condition(s).

Using devices of the type disclosed herein, cells can be maintained under conditions which replicate those in vivo conditions which are normally experienced by said cells. In this way, cells can be cultured or maintained under conditions which ensure that the cells behave and respond substantially as they would in vivo. Further, the cells can be stimulated or exposed to exogenous agents (drugs and the like) and any response determined to be one which is indicative of an in vivo response.

The devices described herein may be used to test the effect of drugs on cells and/or to examine aspects of cell migration, disease process, tumour metastasis/migration, tumour instigation, cell/cell interactions and the like. Again, using a device described herein, the results of such tests can be considered to be generally representative of the results one would see in vivo.

In one embodiment, the device of this disclosure is a fluidic device. A fluid device is a device formed and adapted to permit the flow of fluid into and through the device.

A device according to this disclosure may contain or define fluid carrying channels or conduits. The contained or defined channels or conduits may be arranged to carry fluid to and/or through certain parts of the device.
“”

The device may be a chip-“”

microscope slide and be built on or around a microscope compatible slide.

The channels and/or conduits defined by a microfluidic device or microfluidic chip may have a diameter in the order several micrometres. For example, defined channels and/or conduits may define an internal diameter of about 5 μm to about 30 μm (for example 10 μm, 15 μm, 20 μm, 25 μm or 30 μm).

The devices described herein may be particularly suited to the culture of stem cells and in particular, cancer stem cells (CSC). The cancer stem cell (CSC) is a tumour cell characterised by an ability to self-renew; they may also instigate or initiate clonal tumours and have clonal long-term repopulation potential.

Depending on the type of cancer, the specifics of any given CSC population or cell type, may vary. Devices of this disclosure may be used to grow or maintain any type of CSC including for example, breast cancer CSCs, bowel cancer CSCs, melanoma CSCs, Glioma CSCs, pancreatic cancer CSCs and many others. Cells for use with the devices described herein may be isolated (or provided) directly from a tumour tissue sample. One advantage of this approach is that the device can be personalised and representative of a particular and specific cancer scenario. This ensures that the device is particularly useful for the investigating the specifics of any particular cancer and the effect of certain test reagents on that cancer. In vivo CSCs reside in niches; these are specialised microenvironments which (without wishing to be bound by theory) may help maintain the CSC phenotype. A niche may comprise a mix of environmental conditions (such as temperature, pH, gas level and the like) and companion cell types. For example, a CSC niche may comprise fibroblastic cells, immune cells, endothelial and perivascular cells or their progenitors, components of the extracellular matrix (ECM) components and networks of cytokines and growth factors. The CSC niche may be regarded as part of the tumour microenvironment (TME). Non-CSC tumour cells may also be part of the CSC niche. The progression of any given tumour may depend on the niche and therefore any device which can be used to culture or maintain CSCs under conditions which generally or substantially replicate one or more aspects of these specialised microenvironments can provide a valuable insight into the role of the niche in cancer development, prevention and/or treatment.

Thus, a device of this disclosure may be designed to culture CSCs within an environment that replicates all or part of the in vivo (micro)environment of a CSC and/or one or more aspects of the CSC niche.

The device may comprise or define one or more region(s) for the growth, maintenance or culture of a cell. The (or these) region(s) may (each) take the form of a chamber or well for the growth, maintenance or culture of a cell. The chamber (or well) may be suitable for the growth, maintenance or culture of cancer stem cells (CSCs) and/or any cell which is considered to contribute to a niche to be fully or partially replicated within the device.

For convenience, a well or chamber for the growth maintenance and/or culture of a cell (for example a CSC) will be referred to hereinafter as a “”

The culture chamber(s) may be located within (and defined by) the base part of the device.

One or more, for example each, culture chamber may be in fluid communication with another growth chamber of the device.

The culture chamber may comprise or be defined by poly(methyl methacrylate) (PMMA).

The culture chamber may be lined with a matrigel microstructure.

In use, cancer stem cells (CSCs) may be added directly to the culture chamber of the device.

Cancer stem cells (CSCs) may be applied by printing to any part of the device, including to

‘-’

bio-inks may mimic the extracellular matrix environment and provide support for cell functions such as adhesion, proliferation, and differentiation after printing. It should be appreciated that bio-ink formulations may vary depending on the CSCs type. As stated, the device may define one or more channels or conduits that are in fluid communication with the growth chamber(s) and which may be used to flow substances and/or compositions into and out of the growth chamber(s).

The channels or conduits may define inlet and outlet ports to/from the device. These ports may be used to permit the flow of substances and/or compositions, for example cells, media and/or other substrates, into, through and from the device and in particular into, through and from the culture chamber. For example, cells (for example CSCs) for culture within the culture chamber of the device may be flowed into and out of the culture chamber using the defined channels/conduits.

The inlet and outlet ports may be located at the end of the conduits and/or channels defined by the disclosed device.

Substances or compositions (for example composition comprising cells, media or other substrates) may be flowed into a device of this disclosure using a (micro)fluidic control system such as, for example, the MFCS™-EZ system. For example, cancer associated fibroblasts (CAFs) and cancer stem cells (CSCs) may be fed into the device (and ultimately into the culture chamber) via these microfluidic channels.

The device may comprise or define one or more other wells or growth chambers which are connected to (i.e. in fluid communication with) the culture chamber for CSCs.

These wells or growth “vascular mimetic microfluidic chambers (VMMCs)” one, two or more (for example three, four, five, six, seven or more) VMMCs.

Each VMMC may comprise or be defined within a PDMS substrate.

In one embodiment, the device defines one or more of the VMMC(s).

One or more (for example each) VMMC may comprise a hydrogel microstructure. One of skill will appreciate that a hydrogel may be used to support (the growth and/or maintenance of) cells within the VMMCs.

One or more of the VMMCs may comprise cells. For example, one or more of the VMMCs may comprise tumour associated inflammatory cells (TAICs) and/or endothelial cells.

One or more of the VMMCs may comprise poly(ethylene glycol) diacrylate (PEGDA). This hydrogel may be used to support the growth and/or maintenance of cells within the VMMCs. Where the VMMCs contain endothelial cells and TAICs, the PEGDA may be used to support the growth and maintenance of these cells within the VMMC.

One or more of the VMMCs may comprise a bilayer microvalve—these may be controlled by pressure and can be used to separately and/or independently block the flow of material (fluid, cells, substrate and the like) from the VMMCs into the culture chamber. One of skill will appreciate that this separate and independent control of the flow of material from the VMCCs, allows the user to determine or study the effect of these materials on the cells cultured within the culture chamber. For example, the placement of a bilayer microvalve within a VMMC comprising endothelial cells, will permit the user to separately isolate that VMMC from the growth chamber thus restricting or preventing the flow of material (for example metabolites and other products) from the TAICs and/or endothelial cells contained within the VMMC to the culture chamber. In this way the user can study or determine the effect of these cells (and/or their metabolites) on the culture of cells within the culture chamber.

The device may further define one or more VMMCs comprising polymethyl methacrylate (PMMA). A device of this disclosure may comprise one, two or more (for example three, four, five, six, seven or more) of these PMMA VMMCs.

Each PMMA containing VMMCs may be in fluid communication with the growth chamber.

One or more (for example each) PMMA containing VMMCs may comprise a hydrogel microstructure.

As stated, the device may model or replicate (or emulate or simulate) one or more aspects of the physiological or environmental conditions found in vivo, for example the “ ” conditions within a particular tissue, organ or structure. The device may replicate one or more aspects of the physiological and/or environmental conditions found within a tumour. In other words, the devices provided by this disclosure may replicate (ex vivo) one or more in vivo environmental and/or physiological condition(s), which one or more in vivo environmental and/or physiological condition(s) contribute to what one of skill might refer to as a niche microenvironment. Hereinafter, these replicated physiological and/or environmental conditio “”

The niche may be replicated within one or more parts of the device. For example, the niche may be replicated within one or more of the growth chambers of the device. Where the device is formed and adapted for the growth, maintenance and/or culture of CSCs (within, for example a growth chamber for CSCs) the device may replicate one or more aspects of the CSC niche.

Any given niche may be created by modulating or controlling one or more factors within the disclosed device. These controlled factors may be modulated and/or controlled specifically within the culture chamber of the device. Such factors to be modulated or controlled may include, for example, one or more environmental type factors such as, for example one or more of a temperature, pH, pressure, oxygen content (gaseous ratios) and the like.

By modulating and/or controlling a particular environmental factor within a device of this disclosure (and in particular within the culture chamber of the device) so that it (or they) matches/match the parameters of a corresponding in vivo niche, the devices provided by this disclosure can replicate or simulate the features and/or characteristics of certain or specific in vivo niches (as described above).

A niche to be replicated or simulated within a device of this disclosure may comprise hypoxic conditions. By way of example, the device may comprise an oxygen absorbing agent—which agent reduces the total oxygen content within the device. The oxygen absorbing agent may be applied to one or more surfaces of the device.

An oxygen absorbing agent may be applied to one or more surfaces of the chamber of the device.

Where the device is provided for the purpose of culturing CSCs, the establishment of a hypoxic environment is preferable as this helps maintain the CSCs. “”—that is a defined or distinct region of the chamber may be used to induce or create hypoxic conditions on (or within) the chip. The oxygenator may comprise a series of concentric channels, the surfaces of which are at least partially lined or coated with an oxygen absorbing agent. The oxygenator chamber may be in fluid communication with at least one of the growth chamber(s). For example, the oxygenator may be in fluid communication with the chamber for the growth of CSCs and as such it may regulate the oxygen content of said growth chamber such that the oxygen profile replicates or simulates the oxygen profile of a CSC niche.

One or more of the aforementioned VMMC(s) may connect the culture chamber to the oxygenator chamber. The VMMC(s) linking the oxygenator part of the device to the culture chamber may comprise tumour cells and TICs.

One or more of the VMMCs may further comprise, for example be lined or coated with, an oxygen absorbing agent.

Additionally, or alternatively, the devices described herein may exploit one or more cell types in order to further create, replicate or simulate a particular (in vivo) niche. Accordingly, a device according to this disclosure may comprise one or more cells, which cells are distinct “”hereinafter as the “niche contributing cells”—as these cells (including any excreted or metabolic products thereof) contribute to the niche that is established within the device.

The niche contributing cells may coat or line a surface of the device.

The niche contributing cells may coat or line a surface of a chamber for the growth of cells. The niche contributing cells may line one or more of the defined (micro)channels or (microconduits).

Useful niche contributing cells may include one or more cell types selected from the group consisting of:
(i) Cancer-associated fibroblasts(CAFs)
(ii) Mesenchymal stem cells(MSCs)
(iii) Tumour-associated inflammatory cells
(iv) Non-CSC tumour cells; and
(v) Endothelial cells.

The niche contributing cells may be contained or comprised within one or more of the VMMCs described herein. For example, where the microfluidic device of this disclosure comprises two VMMCs, one may contain endothelial cells and tumour associated inflammatory cells (TAICs). The other VMMC may comprise tumour cells and Tumour initiating cells (TICs).

In view of the above, a device of this disclosure may comprise a growth chamber (for culturing cells, including CSCs), an oxygen absorbing agent and one or more cells selected from the group consisting of:
(i) Cancer-associated fibroblasts (CAFs)
(ii) Mesenchymal stem cells (MSCs)
(iii) Tumour-associated inflammatory cells
(iv) Non-CSC tumour cells; and
(v) Endothelial cells.

In this embodiment, the oxygen absorbing agent may be used to modulate the oxygen content of the growth chamber. For example, the oxygen absorbing agent may line a surface of a region of device (the oxygenator region) which is in fluid communication with the growth chamber.

A device of this disclosure may further comprise one or more components of extracellular matrix (ECM). For example the device may comprise one or more of the components selected from the group consisting of:
(i) collagen
(ii) elastin
(iii) enzymes; and
(iv) glycoproteins (for example fibronectin, laminin).

By way of example, a device that is to replicate the particular niche within a defined tumour tissue may be used to maintain or culture the appropriate CSCs in an oxygen content which is typical or representative of that tumour and/or in the presence of one or more of the cell types that make up the niche, the tumour, its vasculature, structure and surrounding tissue and the like.

A device of this disclosure may comprise
a chamber for the growth of cancer associated fibroblasts and CSCs;
a vascular mimetic chamber comprising tumour associated inflammatory cells and endothelial cells;
an oxygenator in fluid communication with the chamber; and
a vascular mimetic chamber disposed between the chamber and the oxygenator comprising tumour cells and tumour initiating cell.
In this embodiment, the oxygenator may comprise an oxygen absorbing agent. Further, the device may comprise or define channels and/or conduits which permit the flow of substances and/or compositions into, through and from the chamber for the growth of cancer associated fibroblasts and CSCs. The device may be further adapted and formed for connection to a microfluidic flow control system. One of skill will appreciate that a microfluidic flow control system may be used to permit the flow of substances and/or compositions (including cells) into through and from the device (including into through and from the growth chamber part of the device).

The various devices disclosed herein may be made using a variety of techniques. Parts of the device may be made by a 3D printing technique.

The device may comprise a base. The base may be made by a 3D printing technique.

The base part may be biocompatible.

The base may comprise polydimethylsiloxane (PDMS). As a substrate, PDMS is useful as it is biocompatible.

The device may further comprise an elastomeric component.

The elastomeric component may be layered or coated upon the base part.

The elastomeric component may comprise an elastomeric PDMS.

The elastomeric component may be made by 3D printing

The elastomeric component may further comprise mesenchymal stem cells (MSCs). The MSCs may be applied to the elastomeric component before or after it is layered or coated onto the base part of the device.

The MSCs may be provided as a solution in liquid culture. The solution may further comprise “” “”

The elastomeric component may be coated or functionalised. The coating or functionalisation may facilitate cell adhesion. The elastomeric component may be coated or functionalised with, for example alkane thiolates and/or gold nanoparticles. Functionalisation may further comprise the use of adhesive peptides, including, for example, peptides comprising the amino acids Arg-Gly-Asp. Amino acids/peptides of this type can facilitate stem cell adhesion.

Any hydrogel or matrigel component to be used in the manufacture of a device of this disclosure may be manipulated using electron-beam lithography and/or optical lithography. For example matrigel, which in one embodiment is used in the manufacture of a VMMC chamber (using PMMA) may be microfabricated using optical lithography.

The present disclosure also provides an array of devices as described herein. For example, the disclosure may provide a substrate which comprises a plurality of the devices of this disclosure. Each device in the array may be for the culture and maintenance of a particular cell type. Each device in the array may be independently and separately controlled and regulated. For example, a user may be able to selectively and individually alter the conditions in any one of the devices of the array. This allows the user to ensure that each device delivers and maintains the predetermined (micro)environment/niche which is specific to the cell type which is being grown and maintained in the region for growth (the growth chamber) of each of the devices in the array.

The devices of this disclosure may be used to permit the testing of agents, for example drugs, on cells. The devices allow a user to monitor and determine the response of a cell to a test agent or drug. An advantage of the devices disclosed herein is that by maintaining cells in (micro)environments/niches which replicate aspects of the in vivo (micro)environments/niches, the cells will respond to the test agents in a way that better represents how the cells might respond to those test agents/drugs in vivo.

Accordingly, the disclosure provides a method for testing the effects of a test agent or drug on a cell, for example a cancer stem cell, said method comprising, providing a device of this disclosure, maintaining a cell (e.g. CSC) within said device, contacting the cell with a test agent and determining the response of the cell (e.g. CSC) to the test agent.

### DETAILED DESCRIPTION

The present disclosure will now be described by reference to the following figures which show:

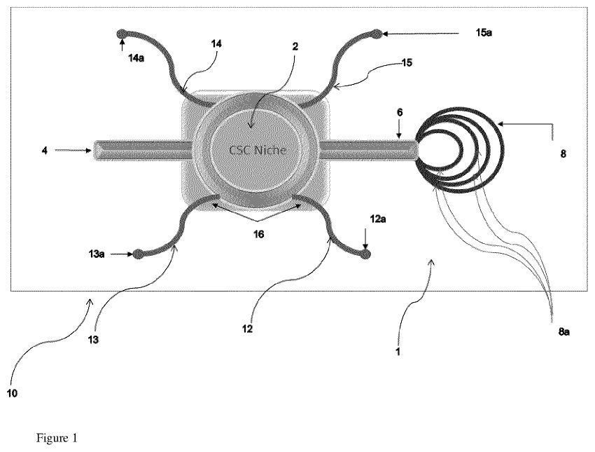

FIG. 1: Schematic diagram of a device according to an embodiment of this disclosure.

The device (10) of FIG. 1 is a microfluidic device for the culture of cancer stem cells (CSCs) within a CSC specific niche. The device (10) comprises a PDMS (polydimethylsiloxane) base (1). Within base (1) there is defined a central chamber for the culture of cells (for example CSCs)—this is shown as feature (2). The CSC niche is established within chamber (2). The device further defines multiple channels and conduits each in fluid communication with the central chamber (2). These channels and conduits are identified as features (4), (6), (8), (12a), (12b), (14a) and (14b) in FIG. 1.

Channel or conduit (4) is a vascular mimetic microfluidic chamber comprising tumour associated cells and endothelial cells. Channel or conduit (4) is in fluid communication with chamber (2).

Channel or conduit (6) is another vascular mimetic microfluidic chamber (manufactured of PMMA) and comprising tumour cells and tumour initiating cells. Channel or conduit (6) is in fluid communication with chamber (2).

Channel or conduit (4) is also in fluid communication with part (8) which is an oxygenator. Oxygenator (8) comprises an oxygen absorbing agent which creates conditions of hypoxia. In this embodiment oxygenator (8) comprises a series of concentrically arranged and generally annular channels (8a). An inside surface of one or more of these channels (8a) may be lined with an oxygen absorbing agent.

Fluid communicating channel/conduit (6) is disposed between chamber (2) and oxygenator (8). In this way, oxygenator (8) controls oxygen levels within channel/conduit (4) and in chamber (2).

Chamber (2) is also connected to inlet conduits (12) and (14) and outlet conduits (13) and (15). Each inlet conduit also has an inlet port (12a and 14a) which permits the addition of substances or compositions into conduits (12) and (14). Inlet conduits (12) and (14) are in fluid communication with chamber (2) and substances or compositions added to these inlet conduits ((12) and (14)) can flow therethrough and into chamber (2). Outlet conduits ((13) and (15)) are also in fluid communication with chamber (2) and substances or compositions within chamber (2) can flow therefrom and out of outlet ports ((13a) and (15a)). In this way, cells, for example CSCs, can be added to chamber (2) by passing them in through inlet port (12a) and allowing them to flow thorough conduit (12) into chamber (2). Additionally, cells such as cancer associated fibroblasts can be added to chamber (2) by passing them in through inlet port (14a) and allowing them to flow through conduit (14) and into chamber (2). Flow of substances and /or compositions into and through and out from conduits (12), (13), (14) and (15) can be facilitated by a microfluidic flow control system such as, for example, the MFCs™-EZ system.

FIG. 1 also shows that device (1) comprises region (16) which is a region of elastomeric PDMS printed as an array onto the PDMS base (1). This region is applied by microcontact printing and contains stamped (for example distinct or discrete) regions of mesenchymal stem cells (MSCs) and extracellular matrix (ECM). If needed, this region can be modified to give rigidity, stretchability and stiffness to the device (10).

The devices described herein create an in vivo like microenvironment for cancer stem cells. The devices are provided as a chip and combine microfluidics and cancer stem cell niches.

In one embodiment, the device is a microscope-slide size microfluidics chip which may comprise:
1. Soft lithographic method used for a patterned PDMS (polydimethylsiloxane) as a substrate on a silicon mould (forming a base for the device)
2.
can be modified to give rigidity, stretching and stiffness required for a CSC niche
3. Matrigel (a hydrogel that can be used to support CSCs) used for providing a 3D microenvironment
4. Oxygenator to create hypoxia and mimic in vivo conditions
5. Branched micro-channels with micromixers which contain the CAFs and can help in cell signalling due to gradient formation.
6. Microfluidic arrays (chambers) with non-CSC tumour cells
7. Vascular mimetic microfluidic chambers (VMMC) lined with endothelial cells and tumour-associated inflammatory cells

In use, the PDMS substrate forms a biocompatible base for the chip.

The device may comprise a layer of MSCs with ECM pools required for a CSC niche stamped onto the layer of PDMS substrate.

An oxygenator helps maintain the hypoxic conditions required for the chip and the PDMS microarray posts can be adjusted to maintain stiffness.

Matrigel which is a hydrogel, is used for cell support and branched micro-channels with mixers bring in the CAFs.

The centre of the chip consists of the microfluidic array chambers as well as the VMMC providing the tumour cells, endothelial cells, and tissue-associated inflammatory cells all required to maintain the niche.

Cancer stem cells can be added to the central growth chamber where they can be maintained in this niche, like in an in vivo environment. The chip can be made compatible for live imaging under the microscope.

In terms of advantages, there are currently a limited number of cancer stem cell models and they do not focus on the CSC microenvironment. The devices that are described herein provide an in vivo like CSC niche which not only helps the user understand the mechanisms of tumour initiation, but can also be used for drug testing, chemotherapeutic testing and in the study of cancer biology. The chip can be extended to make it feasible for diagnostics and can be used for stem cell therapeutics especially for cancers.

Obtaining Cells and Niche Components for a Device According to this Disclosure

As a non-limiting example, the table below details the components required to set up a device according to this disclosure of the culture of breast cancer stem cells (BCSC) within a breast cancer niche.

| | | | | Administration |
|---|---|---|---|---|
| Cell type | Isolation | | Confirmation | into the device |
| Breast | Using tumour tissue | Using | Spheroid | Two inlet ports |
| cancer stem | samples. By flow | peripheral | formation assay | which allow the |
| cells (BCSs) | cytometry, BCSs are | blood - | using | BCSCs to be |
| | characterised by the | flow | MammoCult | loaded into the |
| | presence of the | cytometry | medium. | device using a |
| | following surface | may be | Tumourigenicity - | microfluidic flow |
| | markers: | used by: | testing with | control system |
| | CD44⁺/CD24^{-/low} Lin⁻ | Identifying | mouse xenograft. | (for example |
| | phenotype; | BCSC | Resistance to | MFCs ™-EZ) |
| | CD133; | surface | chemotherapeutic | |
| | CD44⁺CD49f^{hi}CD123/2^{hi} | markers | agents | |
| | phenotype; | seen only | | |
| | CD49f; | in | | |
| | ALDH1; and | circulation | | |
| | CD61. | which | | |
| | By qtPCR, genotype | might | | |
| | characteristics are: | include | | |
| | Like TMX2 (thioredoxin- | CD44, | | |
| | related transmembrane | ANTXR1, | | |
| | protein 2), FAM155B | ALDH1 | | |
| | (family with sequence | and | | |
| | similarity 155, member | CXCR4 | | |
| | B), PTGER3 (prostaglandin | | | |
| | E receptor 3 (subtype | | | |
| | EP3)); | | | |
| | GPR3 (G protein- | | | |
| | coupled receptor 3); and | | | |
| | DDX49 (DEAD | | | |
| | (Asp-Glu-Ala-Asp) box | | | |
| | polypeptide 49). | | | |
| | Analysis may be | | | |
| | completed by magnetic- | | | |
| | activated cell sorting. | | | |

| | | | |
|---|---|---|---|
| Tumour cells | Use biopsy samples and/or tumour | Tumourigenicity | Lined in one of |
| | isolation kits | and proliferation | the VMMCs |
| | | testing | with Matrigel to |
| | | | support growth |
| CAFs | FACS (fluorescence activated cell | RT-PCR | Loaded into the |
| | sorting) method used with -SMA | identifying genes | chip using a |
| | and PDGFR as a surface marker. | like Actin, Oct4, | microfluidic flow |
| | | Nanog and Sox2 | control system |
| | | | (MFCS ™-EZ) |

| | | |
|---|---|---|
| MSCs | Commercially available MSCs | Used as inks on |
| | | PDMS |
| | | elastomeric |
| | | stamps for the |
| | | chip |
| Tumour | Use isolation kits for neutrophils, Treg, macrophages and | Lined in the |
| Associated | MDSCSs | second VMMC |
| inflammatory | | with PEGDA |
| cells | | |
| Tumour | Commercially available HUVECs (human umbilical vein | Lined in the |
| endothelial | endothelial cells) | second VMMC |
| cells | | with PEGDA |
| | | hydrogel to |
| | | support growth |

The various cell types may be quantified using automated cell counting machines (Vi-CELL, cell counter by Thermo Fisher) which counts the cells and also tests the viability of the cells in seconds (Dobbin & Landen, 2013). All the cells involved in the formation of a BCSC niche can be isolated and continually cultured for multiple chips. The niche cells may be expanded using an instrumented stirred vessel, which is a bioreactor for breast cancer cell lines (King & Miller, 2007). A perfusion incubator may be used to help control culture and temperature conditions of the BCSC niche chip.

### Testing a Niche Assembly

With reference to a breast cancer niche assembly (used as a non-limiting example), after the isolation of the niche components, it is important to investigate whether the niche components will self-organise; this can be achieved by the use of monolayer cultures (2D). The culture plates may be pre-coated with Matrigel and PEGDA hydrogel which help provide the optimal growing conditions. The tumour cells, CAFs, MSCs, tumour-associated inflammatory and endothelial cells may all be added in this 2D culture plate. The BCSCs may be added with a micropipette and the cells are cultured with the niche components on a monolayer. This permits simulation of the niche environment and an understanding of its effect on BCSC proliferation and differentiation. Confocal immunofluorescence microscopy can be employed to confirm the niche assembly. The cells are then left to grow for two days and then confirmatory tests and apoptosis flow cytometry can be performed for each niche component and tumour cells.

Precision-cut slice explants: This involves cutting the tumour tissues into very thin slices and embedding them in agarose solutions. This is done by using a tissue embedding unit which allows the agarose to form gel like cylinders (Mitra, et al., 2013). Growth medium along with the niche components can be added and the assembly can be analysed by apoptotic studies and cell viability counts for each niche cell type.

### Quality/Viability of the Device

After the successful fabrication of a device according to this disclosure and following addition of the necessary cells, the entire device can be tested for quality and viability assurance. In one embodiment, this may be achieved by

| SI no: | Quality assurance testing | Expected results |
|---|---|---|
| 1 | Computer simulation (see | The fluid flow, analyte |
| | note 1 below) using a | transport and the overall |
| | software like COMSOL. | function of the chip is |
| | | simulated via the computer |
| | | software, providing a blue |
| | | print of the chip before its |
| | | fabrication (Bedekar, et al., |
| | | 2007). After the simulation, |
| | | a detailed report of the |
| | | results is obtained (see note |
| | | 1 below) |
| 2 | Fault modelling (see note 1 | Test patterns created to |
| | below ™-EZ) | keep the defects to a |
| | | negligible number |
| 3 | Feedback generation (see | Flow checks: Binary codes |
| | note 1 below) using | of 1 and 0 for flow |
| | ™-EZ) | respectively |
| 4 | Experimental testing using a | Time required for flow |
| | software like MATLAB (see | through the chip using an |
| | note 1 below) | actual flow medium |

Note 1:
experiments for quality assurance

1. Computer Simulation of the Entire Chip.

Before testing the chip physically, a computer simulation of the chip is completed. Software is used to produce a detailed design of the entire chip, including its biological components, and used to determine the viability of the chip before it is fabricated. The microfluidic network analysis or simulation is usually divided into four main parts:
involves creating components of the chip
geometry, size, dimensions of the chip and its components
visualization—performance of the chip is simulated and the results are visualized.
uses various modelling techniques for a rapid simulation of the chip, including the biological flow of cells, interaction, signalling etc. with the number of cells administered in the chip (Bedekar, et al., 2007).

The fluid flow, analyte transport and the entire working of the chip can be simulated using computer software, providing a blue print of the chip before its fabrication (Bedekar, et al., 2007). After simulation, a detailed report of the results is obtained. This software also facilitates changes in the dimensions, number of cells administered, flow angles of the microchannels etc. (Bedekar, et al., 2007). Software such as COMSOL, can help with the microfluidic chip simulation virtually testing the BCSC niche on a chip.

2. Fault modelling: Defects in a microfluidic chip can be plenty, ranging from fabrication errors, environmental reasons, leaking of bio-samples, faults in PDMS, misalignment etc. These are rectified by a process called fault modelling (Kai, et al., 2014). This keeps a check on such faults and creates a test pattern that could help keep the defects to a negligible number (Kai, et al., 2014).

3. Feedback generation: This involves testing flow based microfluidic biochips which have inlet ports and outlet ports (Kai, et al., 2014). The VeloChip is a flow based chip, so the inlet ports of the chip are connected to a test set up which includes a digital testing device (binary codes of 1,0). This test set up can help generate feedback with pressure sensors connected to two inlet ports on opposite sides (Kai, et al., 2014). If flow occurs between one region and another, (Kai, et al., 2014). If the test ‘’the flow between one inlet port and another is blocked (Kai, et al., 2014). These procedures can be used to generate measurable feedback in relation to any of the inlet/outlet ports and the flow therebetween.

4. Experimental testing: This involves the use of a flow medium through the microchannels of the chip for testing. The flow medium used to test a PDMS based microchip can be air (Kai, et al., 2014). Advanced pressure sensors can be used to check for the flow rate, the time required for the flow as well as the cell cultures required for the chip (Kai, et al., 2014). The pressure sensors are connected to a software like MATLAB which helps measure and record the time and flow, as well as the culture and maintenance (Kai, et al., 2014)

### Design of Microfluidic Chip Models for 3D Printing

The devices of this disclosure may be used to culture CSCs in a manner which maintains those cells is an environment which replicates aspects of the in vivo environment. This ensures the cells behave and respond in a physiological relevant way.

The devices described herein may be used to test the effects of certain test agents and drugs on CSCs. Indeed, using a device of this disclosure, the response of CSCs to said test agents or drugs can be monitored, studied and/or determined. Gain, because the cells are ‘in vivo environment, the response of the cells to a test agent or drug will be close to or substantially mimic the response one would expect to observe in vivo.

Devices of this disclosure may comprise a base structure.

The base structure may take the form of a microfluidic chip.

The base structure may comprise a set of micro channels.

These micro channels may be fluidically connected to a growth chamber of the device and be used to supply and remove nutrients and/or other factors to/from said growth chamber. In this way, the user is able to maintain a specific environment or conditions within the growth chamber and/or around the maintained CSCs within said growth chamber.

The base structure may comprise a thermoplastic or hydrogel.

The microfluidic channels may be defined by the base structure and/or any thermoplastic or hydrogel component thereof.

The devices of this disclosure (and at least the base part thereof) are biocompatible.

The base structure may comprise a surface upon which cells, for example CSCs can be printed.

The design and manufacture of a device according to an embodiment of this disclosure may Follow the Example Protocol Outlined Below:

### Example Design Protocol

This example procedure may allow for the design of a robust and 3D printable design file for a microfluidic chip for cancer environment drug testing.

PROTOCOL (note all measurements and parameters are for example only and may be varied).

1) Ensure access to a 3D parametric CAD modelling software (ideally one of the suggested in the materials section).

2) Consider the design specification outlined below:
Biocompatibility with the cancer cell environment
Printable by the available 3D printer
Dimensions of part must not exceed printer dimension capacity (In the case of the inkredible this is 120×80×70)
Must contain:
   2×Inlet channel
   2×Outlet channel
   1×Vascular mimetic microfluidic chamber (for printing the environment)
   CNC niches (wells to contain cancer cells)
Manufactured from biocompatible material
Plate thickness of 5 mm
Channels must have diameters a minimum of the resolution of the printer
Using the specification define the channel diameter, ideally this value should fall between 0.1- and 0.3-mm diameter.
Define the diameter of each well, values previously used have been ¾ mm

5) Define the number of wells needed in the chip, previous plate designs have had 1, 25 (5×5) and 96 (8×12) wells. From this value and the dimeter of each well calculate plate width and height leaving a suitable distance between each well (approx. 2 mm)

6) Define is there will be a method of oxygenation and if it will be via a spiral channel [x], a channel over the well surface [y] or an alternative method.

7) Open the

8) Extrude a block of your determined width and height and (for example) 5 mm depth

9) Sketch the centre of the top left well and bottom right wells on the face of the block and revolve cut a semi-circle through 180 degrees to make the top left well.

10) ‘’centre of the bottom right.

11) Create the vascular mimetic microfluidic chamber to connect the rows of wells by cutting the surface

12) Add the oxygen gradient feature if required (height may need to be added if a spiral is being used)

13) Save the part. With the title baseplate_[A]×[B]_[C]oxygenated

### Where:

[A]=number of rows
[B]=number of columns
[C]=type of oxygenation ie spiral, surface, not ect.

14) Now to make the top of the plate, open a new isometric part and call it: topplate_[A]'[B]_[C]oxygenated

15) Extrude a block of the same width, height but of (for example) 2 mm depth

16) Create small holes in through the plate to act as inlet and outlet ports so they line up with the first and last wells, create a hole to allow for oxygen to enter the environment that aligns with the selected feature and save the file.

17) Export both files in STL format under the 3D printing tab ensuring the export dimensions are in mm and the resolution is fine.

### Expected Outcome/Result

The expected outcome should be 2 STL files that make up a microfluidic chip model, one for the base and one for the top. This model can then be imported into a slicing software, a material selection can be made by referring to SOP/BM/002 and the chip can be 3D printed. The model should meet the design specification fully and be saved as both .par and .stl files for future reference.

### Selection of Compatible Inks for Microfluidic Chips

### Introduction

A microfluidic device of this disclosure is a small chip that allows liquid to flow through it and has widespread applications in diagnostics and medical testing.

The device may be manufactured using materials that are selected to be printable on a micron scale, biocompatible, stiff, transparent and/or non-biodegradable.

Advantageously, the materials should not interact with the cancer environment.

The device may comprise thermoplastics (for example PMMA, PC, high impact polystyrene and polyethylene terephthalate (PET) and polycaprolactone PCL) which require a heated nozzle to melt the plastic. The device may also comprise hydrogels for example, collagen, gelatine, alginate and/or polysaccharide based hydrogels.

Optimal 3D printable inks that are compatible for printing microfluidic chips may be selected. These may sustain a range of various cancer microenvironments. The operating procedure would allow for an ink to be identified that is compatible with the printer available and suited to its application.

### Example Protocol

If a heated printhead is available that reaches temperatures suitable for melting thermoplastic filament refer to protocol [A] if not, refer to protocol [B].

### [A]

Due to the availability of a heated nozzle a wide range of thermoplastics are available for printing. The most popular biocompatible thermo plastics are compared in the table below alongside associated properties. By considering literature at the time of material selection, further possible materials should be added to the comparison.

| Material | Extruder temp | Properties |
|---|---|---|
| PMMA | 245-255 degrees | U.V resistance |
| | Celsius | Chemical resistance |
| | | 80-93% |
| | | transparency |
| | | Requires heated |
| | | print bed |
| PC | 260-310 degrees | High durability |
| | Celsius | High melting point |
| | | Requires heated |
| | | print bed |
| | | transparency |
| High impact | 220-240 degrees | transparency |
| Polystyrene | Celsius | high impact strength |
| | | poor thermal stability |
| | | required heated print |
| | | bed |
| Polyethylene | 75-90 degrees | fully recyclable |
| terephthalate | Celsius | rigid |
| (PET) | | good chemical |
| | | resistance |
| | | brittle |
| Polycaprolactone | 115-145 degrees | low glass transition |
| PCL | Celsius | temp around 60 |
| | | degrees Celsius |
| | | bioresolvable |
| | | high impact strength |
| | | high durability |

First the availability of a heated print bed needs to be considered, if this is available the higher meting points, glass transition and strength of PMMA, PC or high impact polystyrene make them more suitable options. If not PET or PCL may be used.
If a heated print bed is not available PET should be selected if the microfluidic device is indented to be used for extended periods of time. Cellink provide a compatible PCL bioink but it is bioresolvable so will be broken down by the environment over time making it not ideal for cancer environment testing.
If a heated print bed is available, the maximum extruder temperature then needs to be considered, if higher temperatures are available PMMA or PC are the optimal choice, if slightly lower temperatures up to 250 degrees Celsius can be reached, high impact polystyrene can be used.

### [B]

When no heated printhead is available hydrogels should be printed and then crosslinked. Various hydrogels should be considered to make the optimal selection, the most common are outlined in the table below.

| Material | Notes |
|---|---|
| Collagen | Contain factors that allow for cell adhesion |
| | and proliferation cured thermally or with UV |
| | light. |
| Gelatine | Contain factors that allow for cell adhesion |
| | and proliferation, cured with UV light or/and |
| | crosslinking solution |
| Alginate | Lower compressive strength |
| | Mechanical stiffness largely depends on |
| | alginate concentration. Crosslinked with |
| | crosslinking solution |
| Polysaccharide | Found to have compatibility with skin and |
| | tumour applications. Crosslinked with |
| | crosslinking solution |

If a U.V. light is available a gelatine or collagen-based hydrogel should be selected, if not alginate or polysaccharide would be suitable
If cell adhesion or proliferation is required, Collagen, Matrigel or gelatine-based hydrogels should be considered. The hydrogel used should mimic the natural ECM of the cancer environment as closely as possible yet achieve a high enough stiffness upon crosslinking to support the structure.
After eliminating hydrogels not compatible with available curing mechanisms or cell adhesion requirements, supplier companies should be contacted to establish specific recommendations on the stiffest materials available due to the range of hydrogels on the market with various compositions.

### Expected Outcome/Result

The result should allow for identification of a suitable material for 3D printing microfluidic devices with the equipment available. The material should fill all the required specifications and allow for a suitable cell printing and drug testing surface. In the case of using the Cellink lnkredible printer GeIXA (gelatine methacrylate, xanthan gum and alginate) and was found to be the most suitable material due to the lack of heated printhead and print bed but the availability of a UV light with facilitated cross-linking.

### Selection of Compatible Bioinks for 3D Printing Simple Cancer Environment.

In order to 3D bioprint cancer cell environments, the cells may need to be contained within an artificial matrix which takes the form of a bioink. The bioink acts in place an extracellular matrix (ECM) surrounding the affected tissue. Bioinks can be categorised into four types; structural, functional, sacrificial and non-sacrificial (Cellink 2020). The bioinks used to simulate cancer environments are functional. Those used for 3D extrusion bioprinting are typically hydrogels but a range of various hydrogels are available. Common hydrogels include alginate, gelatin, collagen, fibrin, hyaluronic acid, agarose, chitosan and polyethylene. It has been found that ECM mechanical properties drastically change cell behaviour, particularly stem cell differentiation (Engler et al. 2006) which goes onto impact tumour migration (Albritton and Miller 2017). Furthermore, a stiffer matrix has been correlated to more aggressive and later stage cancers (Gungor-Ozkerim et al. 2018) adding to the list of considerations that need to be made when selecting a bioink for a specific cancer line.

HA based bioinks are most compatible with brain tumour environments whist alginate/RGD combinations have previously been used for printing breast tumour environments.

CELLINK A-RGD (Sodium alginate and RGD) may be used for printing breast cancer lines.

### Purpose

The structure and composition of the ECM within the body varies so the selected bioink should be matched to the ECM in which the specific cancer line is found. Accordingly, cells for use (for example CSCs) may be formulated with a bioink having a composition and properties which match or replicate at least some of the properties (and/or characteristics) of the ECM which usually surrounds the cell type to be used.

For example, a bioink for use may be selected because it has a stiffness that matches the relevant ECM. In one embodiment, when the user has identified the exact cancer line to be bioprinted, the stiffness and the key components of the ECM may be determined and matched to a bioink for use. By way of example, a major component of the bone ECM is hydroxyapatite whilst a major component of skin is collagen. This information can be used to select an appropriate bioink for use.

The bioink may be cross-linked after selection. In this way it may better match the viscosity and/or stiffness of the relevant ECM.

In this way the 3D printed cells remain viable throughout the drug testing process and the various cells present in the cancer environment including mesenchymal stem, inflammatory and cancer associated epithelial can be printed together onto a biocompatible chip.

Materials Required (with Catalouge Numbers/Suppliers)

Suitable cancer cell compatible bioinks (based on hydrogels) may include those outlined in the table below.

| BioInk | Material |
|---|---|
| Collagen PREMIUM | Collagen type I |
| Chitoink | Chitosan |
| ColMa | methacrylated collagen |
| Cell-link Laminink | Laminins-basal lining of cell |
| | membrane |
| GelXA laminink | Same as above but more |
| | stable at room temp |
| GelXG | Gelatin-based with Xanthan |
| | gum |
| Alginate | Alginate |
| GelMA High | gelatin methacrylate |
| GelMA-Alginate | gelatin methacrylate |
| GelMA HA | gelatin methacrylate |
| Gel XA | GelMA base, xanthan gum |
| | and alginate |
| Coll 1 | Collagen type I |
| GelMA A | methacrylated collagen and |
| | alginate |
| GelMA C | |
| Cellink A | Sodium alginate |
| Celllink RGD | Polysaccharide hydrogel and |
| | RGD |
| Cellink A RGD | Sodium alginate and RGD |
| Cellink bioink | Polysaccharide hydrogel |

### Expected Outcome/Result

By selecting biocompatible materials the expected outcome is continuous filament extrusion to build a stable structure prior to crosslinking, long term stability after cross-linking and supported cell viability throughout the entire bioprinting process. As stated, the chosen material should mimic the natural cancer environment and be compatible with the microfluidic device material onto which it is to be printed. By mimicking the natural cancer environment, the printed cells should produce a similar reaction to the cancer drugs as would be produced in the human body with the material either being selected to be of a similar stiffness as the ECM or crosslinked to the stiffness of the ECM after printing.

### REFERENCES

Bedekar, A. S. et al., 2007. Design Software for Application-Specific Microfluidic Devices. Clinical Chemistry, 53(11).
Dobbin, Z. C. & Landen, C. N., 2013. Isolation and Characterization of Potential Cancer Stem Cells from Solid Human Tumors—Potential Applications. Curr Protoc Pharmacol., Volume 63, pp. 1-24.
Kai, H., Yu, F., Ho, T.-Y. & Chakrabarty, K., 2014. Testing of Flow-Based Microfluidic Biochips: Fault Modeling, Test Generation, and Experimental Demonstration. IEEE Transactions on Computer-Aided Design of Integrated Circuits and Systems, 33(10), pp. 1463-1475.
King, J. A. & Miller, W. M., 2007. Bioreactor Development for Stem Cell Expansion and Controlled Differentiation. Curr Opin Chem Biol., 11(4), pp. 394-398.
Mitra, A., Mishra, L. & Li, S., 2013. Technologies for deriving primary tumor cells for use in personalized cancer therapy. Trends Biotechnol., 31(6), pp. 347-354.
Plaks, V., Kong, N. & Werb, Z., 2015. The Cancer Stem Cell Niche: How Essential Is the Niche in Regulating Stemness of Tumor Cells? Cell Stem Cell, Elsevier Inc, Volume 16, pp. 225-238.
Albritton, J. L., and J. S. Miller. 2017. ‘3D bioprinting: improving in vitro models of metastasis with heterogeneous tumor microenvironments’, Dis Model Mech, 10: 3-14.
Engler, A. J., S. Sen, H. L. Sweeney, and D. E. Discher. 2006. ‘Matrix elasticity directs stem cell lineage specification’, Cell, 126: 677-89.

Gungor-Ozkerim, P. S., I. Inci, Y. S. Zhang, A. Khademhosseini, and M. R. Dokmeci. 2018. ‘Bioinks for 3D bioprinting: an overview’, Biomater Sci, 6: 915-46.

1. A device for the culture of cells, which device supports and/or maintains the cells within an environment which mimics one or more in vivo environmental condition(s), wherein the device defines one or more region(s) for the growth, maintenance or culture of a cell.

2. (canceled)

3. The device of claim 1, wherein the wherein the region for the growth, maintenance or culture of a cell is lined with a matrigel microstructure.

4. The device of claim 1, wherein the device further defines fluid carrying channels or conduits arranged to carry or transport fluid to and/or through the region for the growth, maintenance or culture of a cell.

5. The device of claim 1, wherein the device comprises one or more vascular mimetic microfluidic chambers (VMMCs), wherein the or each VMMC is in fluidic communication with the region for the growth, maintenance or culture of a cell.

6. The device of claim 5, wherein the VMMC comprises tumour associated inflammatory cells (TAICs) and/or endothelial cells.

7. The device of claim 5, wherein the VMMC comprises a valve which may be opened and closed to regulate the flow of material and/or metabolites from the TAICs and/or endothelial cells of the VMMC to the region for the growth, maintenance or culture of a cell.

8. The device of claim 1 where the device supports and/or maintains stem cells or cancer stem cells (CSC).

9. The device of claim 1, wherein the cells are breast cancer CSCs, bowel cancer CSCs, melanoma CSCs, Glioma CSCs or pancreatic cancer CSCs.

10. The device of claim 8, wherein the device maintains the stem cells or CSCs within an environment that replicates all or part of the in vivo (micro)environment of the stem cell or CSC and/or one or more aspects of the stem cell/CSC niche.

11. The device of claim 10, wherein the (micro)environment and/or niche is created by modulating or controlling one or more factors within the region(s) for the growth, maintenance or culture of a cell.

12. The device of claim 11, wherein the factors to be modulated or controlled one or more of a temperature, pH, pressure, oxygen content and gaseous ratios.

13. The device of claim 10, wherein the (micro)environment or niche within the region(s) for the growth, maintenance or culture of a cell, may comprise hypoxic conditions.

14. The device of claim 1, wherein the device comprises an oxygen absorbing agent for reducing the total oxygen content within the device and in particular within the region(s) for the growth, maintenance or culture of a cell.

15. The device of claim 1, wherein the device defines a chamber which comprises the oxygen absorbing agent, said chamber being in fluid communication with each of region(s) for the growth, maintenance or culture of a cell.

16. The device of claim 15, wherein the chamber which comprises the oxygen absorbing agent defines a series of concentric channels, the surfaces of which are at least partially lined or coated with an oxygen absorbing agent.

17. The device of claim 1, wherein the device comprises one or more additional cells selected from the group consisting of:
(i) Cancer-associated fibroblasts(CAFs)
(ii) Mesenchymal stem cells(MSCs)
(iii) Tumour-associated inflammatory cells
(iv) Non-CSC tumour cells; and
(v) Endothelial cells.

18. The device of claim 17, wherein the additional cells help maintain a niche or microenvironment for the culture of stem cells or cancer stem cells.

19. The device of claim 17, wherein the additional cells coat or line a surface of the device.

20. (canceled)

21. (canceled)

22. A device comprising a growth chamber for culturing CSCs, an oxygen absorbing agent and one or more cells selected from the group consisting of:
(i) Cancer-associated fibroblasts (CAFs)
(ii) Mesenchymal stem cells (MSCs)
(iii) Tumour-associated inflammatory cells
(iv) Non-CSC tumour cells; and
(v) Endothelial cells.

23. The device of claim 22, wherein the oxygen absorbing agent modulates the oxygen content of the growth chamber.

24. The device of claim 22, wherein the device comprises one or more components of extracellular matrix (ECM).

25. The device of claim 24, wherein the components of ECM are selected from the group consisting of:
(i) collagen
(ii) elastin
(iii) enzymes;
(iv) glycoproteins
(v) Fibronectin; and
(vi) laminin.

26. (canceled)

27. (canceled)

28. (canceled)

29. (canceled)

30. A method of testing the effect of an agent on a cell, said method comprising culturing the cell in a device according to claim 1, contacting the device and cell with the test agent and determining the response of the cell to the test agent.

31. The method of claim 30, wherein the cell is a cancer stem cell.
